(19) United States
(12) Patent Application Publication
(10) Pub. No.: US 2024/0374296 A1
(43) Pub. Date: Nov. 14, 2024

(54) SYSTEM AND METHOD FOR TREATING BONE FRACTURES

(71) Applicant: GLOBUS MEDICAL, INC., AUDUBON, PA (US)

(72) Inventors: David Machamer, Glen Mills, PA (US); Jonan Philip, Phoenixville, PA (US)

(21) Appl. No.: 18/779,368

(22) Filed: Jul. 22, 2024

Related U.S. Application Data
(62) Division of application No. 17/578,745, filed on Jan. 19, 2022, now Pat. No. 12,064,150.

(51) Int. Cl. A61B 17/74; A61B 17/80; A61B 17/84

(52) U.S. Cl. CPC A61B 17/744; A61B 17/746; A61B 17/8061; A61B 17/84

(57) ABSTRACT

An intramedullary nail includes one or more nail openings extending therethrough. The intramedullary nail is implantable within a medullary canal of the bone. A bone plate is configured to engage an outer surface of the bone. The bone plate includes one or more plate openings extending therethrough. The plate openings are spaced about the bone plate such that the bone plate is positionable to axially align the plate openings with the nail openings when the intramedullary nail is implanted within the medullary canal of the bone. An aiming guide includes one or more targeting openings. The targeting openings are spaced about the aiming guide such that the aiming guide is positionable to axially align the targeting openings with the plate openings and the nail openings for extending one or more stabilizing fasteners therethrough when the intramedullary nail is implanted within the medullary canal of the bone.

### CROSS-REFERENCE TO RELATED APPLICATIONS

This application is a divisional patent application of U.S. patent application Ser. No. 17/578,745 filed on Jan. 19, 2022, which is incorporated in its entirety herein.

### FIELD

The present disclosure generally relates to treating bone fractures.

### BACKGROUND

Following an injury to a long bone, such as a fracture of the femur or tibia, one or more fixation devices may be used to immobilize the fracture fragments and stabilize the long bone. Intramedullary nails, for example, may be inserted into the intramedullary canal of the bone and provide the appropriate proximal and/or distal fixation. However, known intramedullary devices may suffer from a number of disadvantages. For example, they may be susceptible to implant failure and difficulty in alignment of a fixation screw with respect to the intramedullary nail.

### BRIEF SUMMARY

The present disclosure enables a fixation screw to be extended at least partially through an intramedullary nail. In one aspect, a system is provided for treating a fracture in a bone. The system includes an intramedullary nail including one or more nail openings extending therethrough for receiving one or more stabilizing fasteners. The intramedullary nail is implantable within a medullary canal of the bone. A bone plate is configured to engage an outer surface of the bone. The bone plate includes one or more plate openings extending therethrough for receiving the stabilizing fasteners. The plate openings are spaced about the bone plate such that the bone plate is positionable to axially align the plate openings with the nail openings when the intramedullary nail is implanted within the medullary canal of the bone. An aiming guide includes one or more targeting openings for receiving the stabilizing fasteners. The targeting openings are spaced about the aiming guide such that the aiming guide is positionable to axially align the targeting openings with the plate openings and the nail openings for extending the stabilizing fasteners therethrough when the intramedullary nail is implanted within the medullary canal of the bone.

In another aspect, an aiming guide is provided for treating a fracture in a bone. The aiming guide includes a body and a pair of legs extending from the body. Each leg of the pair of legs includes one or more targeting openings for receiving one or more stabilizing fasteners therethrough. The targeting openings are spaced about the pair of legs such that the aiming guide is positionable to axially align the targeting openings with one or more openings of an intramedullary nail implanted within a medullary canal of the bone.

In yet another aspect, a method is provided for treating a fracture in a bone. The method includes coupling an intramedullary nail to an insertion tool. The intramedullary nail includes one or more nail openings extending therethrough for receiving one or more stabilizing fasteners. An aiming guide is coupled to the insertion tool. The aiming guide includes one or more targeting openings for receiving the stabilizing fasteners. The intramedullary nail is implanted within a medullary canal of the bone. A bone plate includes one or more plate openings extending therethrough such that the plate openings are axially aligned with the targeting openings. The stabilizing fasteners are extended through the plate openings and the nail openings using the targeting openings.

### BRIEF DESCRIPTION OF THE DRAWINGS

FIG. 1 is a perspective view of an example system for treating a bone fracture, including the insertion tool, an aiming guide, and a bone plate.

FIG. 2 is a perspective view of an example insertion tool for treating a bone fracture, including a handle and an intramedullary nail coupled to the handle;

FIG. 3 is a perspective view of the insertion tool shown in FIG. 2;

FIGS. 4 and 5 are detailed cross-sectional views of the insertion tool shown in FIG. 2 with an example assembly shaft;

FIG. 6 is a side view of the insertion tool shown in FIG. 2 with an example backslap shaft;

FIG. 7 is a side view of the bone plate shown in FIG. 1 coupled to a long bone;

FIG. 8 is a top view of the bone plate shown in FIG. 1 coupled to the long bone;

FIG. 9 is a perspective views of the system shown in FIG. 1 including driver sleeves;

FIG. 10 is a perspective view of another example system for treating a bone fracture, including the insertion tool, the aiming guide, and another bone plate;

FIG. 11 is a side view of the bone plate shown in FIG. 10 coupled to a long bone;

FIG. 12 is a top view of the bone plate shown in FIG. 10 coupled to the long bone;

FIG. 13 is an alternative perspective views of the system shown in FIG. 6 including driver sleeves;

FIG. 14 is a side view of the aiming guide shown in FIG. 1;

FIG. 15 is a front view of the aiming guide shown in FIG. 1;

FIG. 16 is a cross-sectional view taken along line A-A in FIG. 15;

FIG. 17 is a detailed view of circle A in FIG. 16; and

FIG. 18 is a flowchart of an example method for treating bone fractures.

Corresponding reference characters indicate corresponding parts throughout the drawings.

### DETAILED DESCRIPTION

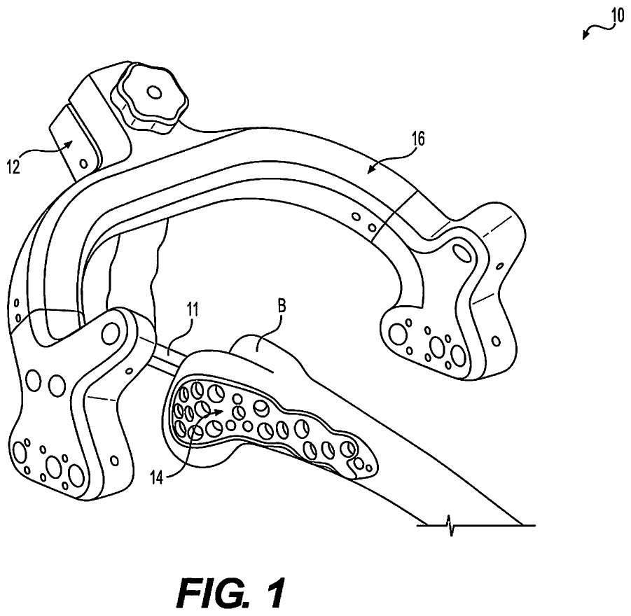

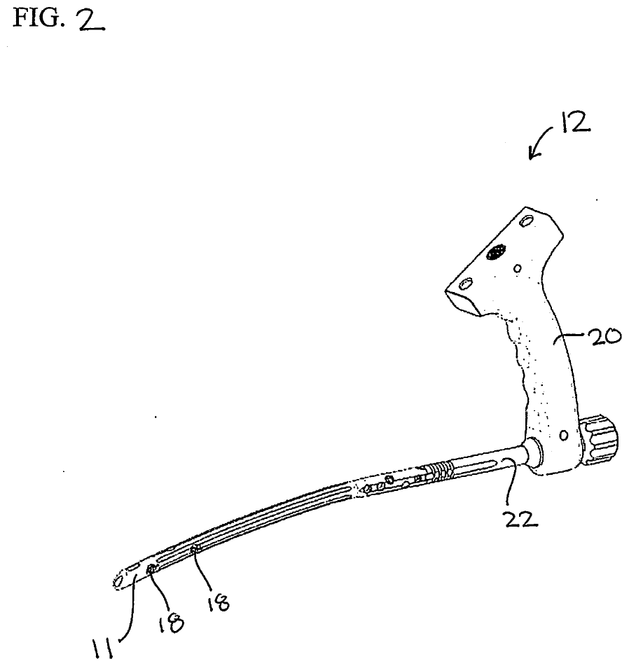

FIG. 1 shows a system 10 for treating a fracture in a bone B. The system 10 may include the intramedullary nail 11 and insertion tool 12, a bone plate 14 (e.g., a washer plate), and/or an aiming guide 16. Referring to FIGS. 2-6, an example insertion tool 12 may be coupled to an intramedullary nail 11 for use in handling or moving the intramedullary nail 11. The insertion tool 12 may be used, for example, to advance, retract, and/or rotate the intramedullary nail 11. In some examples, the insertion tool 12 is used to insert the intramedullary nail 11 into a long bone (e.g., after reaming).

The intramedullary nail 11 includes an elongate body extending between a distal end and a proximal end. In some examples, the intramedullary nail 11 defines a hollow channel extending axially therethrough. Alternatively, the intramedullary nail 11 may be solid along its length. The intramedullary nail 11 is configured to extend axially within the intramedullary canal of a bone. In some examples, the intramedullary nail 11 may be substantially straight. Alternatively, the intramedullary nail 11 may include one or more curves or bends that conform to the anatomical shape of the intramedullary canal.

The intramedullary nail 11 may be comprised of one or more biocompatible materials that enable the intramedullary nail to have sufficient strength to secure and hold bone. For example, the intramedullary nail 11 includes one or more of titanium, cobalt chrome, cobalt-chrome-molybdenum, stainless steel, tungsten carbide, carbon composite, polyetheretherketone (PEEK), polyethylene, ultra-high molecular weight polyethylene (UHMWPE), resorbable polylactic acid (PLA), polyglycolic acid (PGA), and/or an alloy of such materials.

As shown in FIG. 2, the intramedullary nail 11 may include one or more slots and/or nail openings 18 extending therethrough. The nail openings 18 are arranged or spaced to form an opening pattern about the intramedullary nail 11. The nail openings 18 may be configured to receive and guide one or more stabilizing fasteners (e.g., nail, screw, bolt) therethrough. In some examples, one or more nail openings 18 may include one or more threads configured to engage one or more threads of the stabilizing fasteners. Each nail opening 18 may be oriented to have a trajectory defining an angle relative to the longitudinal axis of the intramedullary nail 11. For example, the nail openings 18 may be defined to extend generally radially in the anterior-posterior (AP) and/or medio-lateral (ML) direction. Alternatively, the nail openings 18 may be oriented to have any trajectory that enables the intramedullary nail 11 to function as described herein.

The insertion tool 12 includes a handle 20 and an insertion shaft 22 extending from the handle 20 in a distal direction. The insertion shaft 22 may include one or more orientation tabs 24 that enable the intramedullary nail 11 to be desirably positioned and/or oriented relative to insertion tool 12. As shown in the current example, the insertion shaft 22 has three orientation tabs 24 distally extending from its distal end that aid in ensuring that the intramedullary nail 11 is oriented correctly when the intramedullary nail is coupled to the insertion shaft. For example, the intramedullary nail 11 may be oriented such that the nail openings 18 extend generally radially in the AP and/or ML direction. In some examples, the insertion shaft 22 may include one or more grooves 26 at a distal portion thereof to enable a user (e.g., surgeon) to locate a proximal end of the intramedullary nail 11 (e.g., under fluoroscopy). The grooves 26 may be spaced axially about 0.2 inches (about 5.0 millimeters) apart and/or extend circumferentially about an outer surface of the insertion shaft 22.

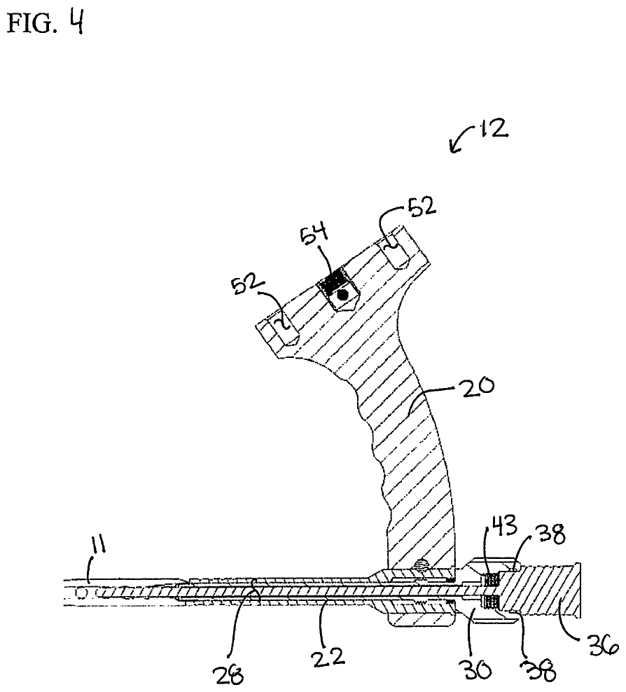

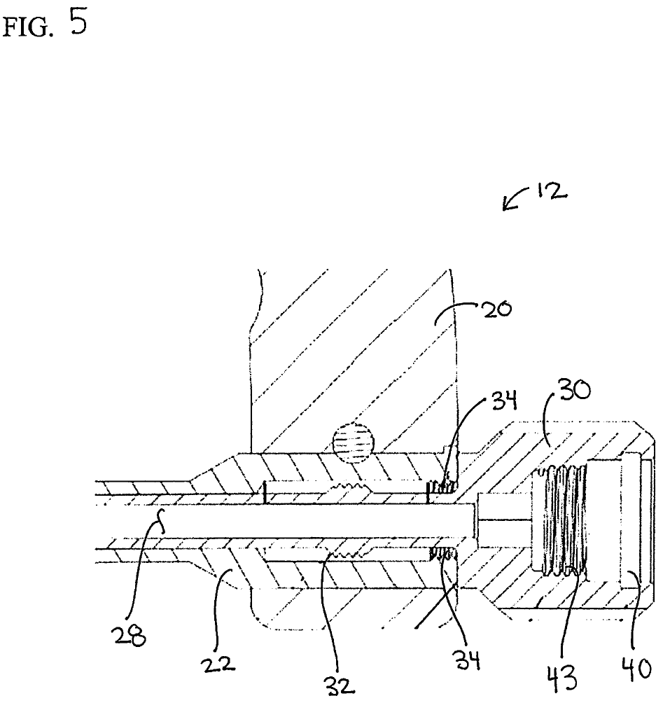

As shown in FIGS. 4 and 5, the insertion shaft 22 may extend at least partially through the handle 20 and/or include a flange at a proximal portion thereof that seats into a groove defined in a proximal surface of the handle 20. In some examples, the handle 20 is fabricated from a composite material, and/or the insertion shaft 22 is fabricated from a stainless steel material. Alternatively, the handle 20 and/or insertion shaft 22 may be fabricated from any combination of materials that enables the insertion tool 12 to function as described herein.

As shown in FIGS. 4 and 5, the insertion shaft 22 may define a cannula or channel 28 extending axially therethrough. A connection bolt 30 may be extended axially through the channel 28 of the insertion shaft 22 to facilitate quick and easy assembly of the intramedullary nail 11 onto the insertion tool 12. For example, the connection bolt 30 may be translated axially to selectively extend a distal end of the connection bolt 30 beyond a distal end of the insertion shaft 22 and into a proximal end of the intramedullary nail 11. When the connection bolt 30 is translated axially to selectively retract the distal end of the connection bolt 30, the distal end of the insertion shaft 22 acts as a stop for the intramedullary nail 11 such that the distal end of the connection bolt 30 may be withdrawn from the proximal end of the intramedullary nail 11.

The connection bolt 30 may include one or more external threads 32 configured to engage or cooperate with one or more internal threads 34 of the insertion shaft 22. As shown in FIGS. 4 and 5, when the external threads 32 of the connection bolt 30 are distal to the internal threads 34 of the insertion shaft 22 and in a cavity defined by the insertion shaft 22 (i.e., the connection bolt 30 is fully threaded into the insertion shaft 22), the threads of the connection bolt 30 and/or insertion shaft 22 are configured to prevent the external threads of the connection bolt 30 from falling out of the cavity while allowing the external threads of the connection bolt 30 to freely translate within the cavity. In this manner, the connection bolt 30 may be configured to translate axially a predetermined distance. In some examples, a user (e.g., surgeon) may strike a proximal portion of the connection bolt 30 to impact the intramedullary nail 11 through the insertion tool 12.

In some examples, an assembly shaft 36 may be used to further aid in the connection of the intramedullary nail 11 onto the insertion tool 12. As shown in FIG. 4, the assembly shaft 36 may be inserted into and through the connection bolt 30 and/or insertion shaft 22. A distal end of the assembly shaft 36 may be selectively extended beyond the distal end of insertion shaft 22 and/or connection bolt 30 and into the channel or cannula of the intramedullary nail 11. In this manner, the assembly shaft 36 may be used to align the intramedullary nail 11 with the insertion shaft 22 and/or connection bolt 30 as the intramedullary nail 11 is coupled to the insertion tool 12 (e.g., connection bolt 30). The assembly shaft 36 may include a latching feature 38 that interfaces with an undercut groove 40 in the connection bolt 30 to retain the assembly shaft 36 once inserted.

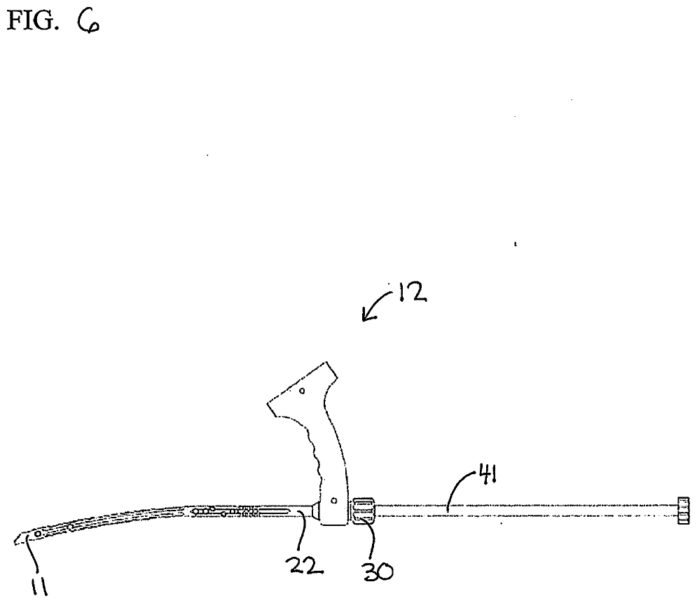

As shown in FIG. 6, the insertion tool 12 may include or be coupled to a backslap shaft 41 for use in retracting the intramedullary nail 11. The backslap shaft 41 may be coupled to the insertion shaft 22 and/or connection bolt 30. For example, as shown in FIGS. 4 and 5, the connection bolt 30 may include one or more internal threads 43 for use in connecting the backslap shaft 41. In this manner, a user (e.g., surgeon) may strike a proximal portion of the backslap shaft 41 (e.g., in a proximal direction) to impact the intramedullary nail 11 through the insertion tool 12.

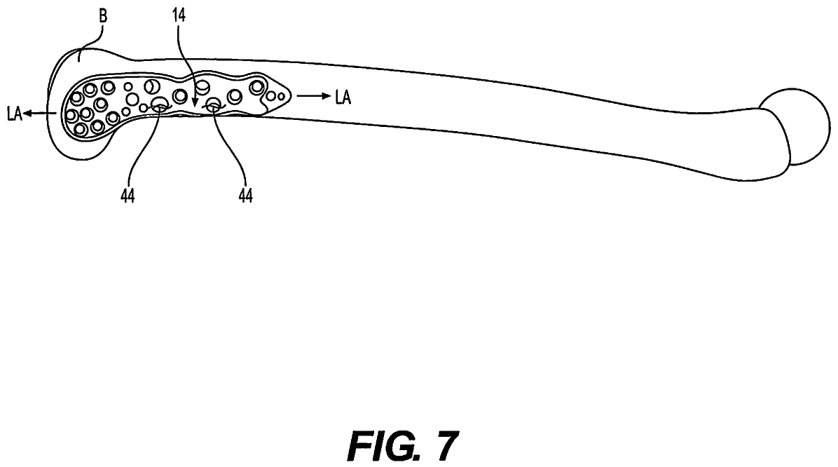

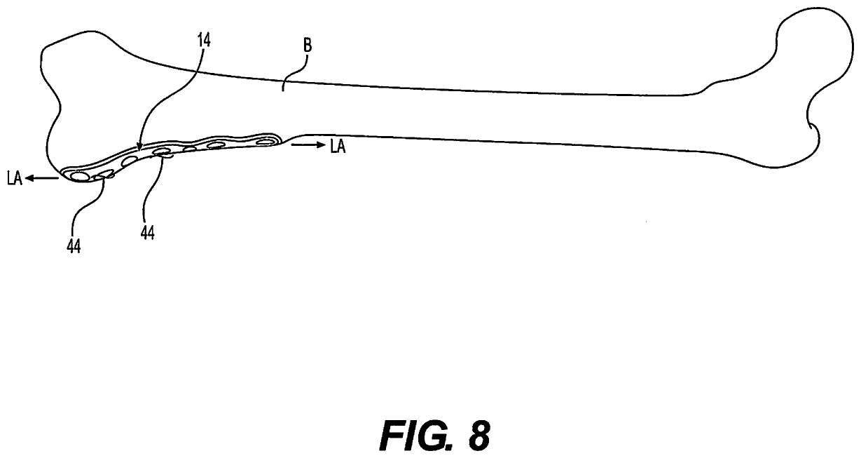

As shown in FIGS. 7 and 8, the bone plate 14 may be oriented to extend generally along or parallel to a longitudinal axis LA of the bone B and positioned on a medial and/or lateral side of the bone B to engage at least a portion of a condyle and/or shaft of the bone B. In some examples, the bone plate 14 is configured and/or contoured to match or lie substantially parallel to a particular outer bone surface, such as an epiphysis region, a metaphysis region, and/or a diaphysis region of the bone B. In this manner, the bone plate 14 may be used to stabilize distal femur fractures as well as distal femur articular fractures as a stand-alone reduction device, a provisional fixation device, and/or a final fixation device.

In some examples, the bone plate 14 may be used with one or more stabilizing fasteners (e.g., nail, screw, bolt) to increase a load carrying capability of the construct. For example, the bone plate 14 may be used with standard locking screws and/or with dedicated washer bolts that compress the condyles of the bone B when tightened and/or provide support to the articular block and femoral shaft. In some examples, the stabilizing fasteners may be extended through one or more nail openings 18 of the intramedullary nail 11.

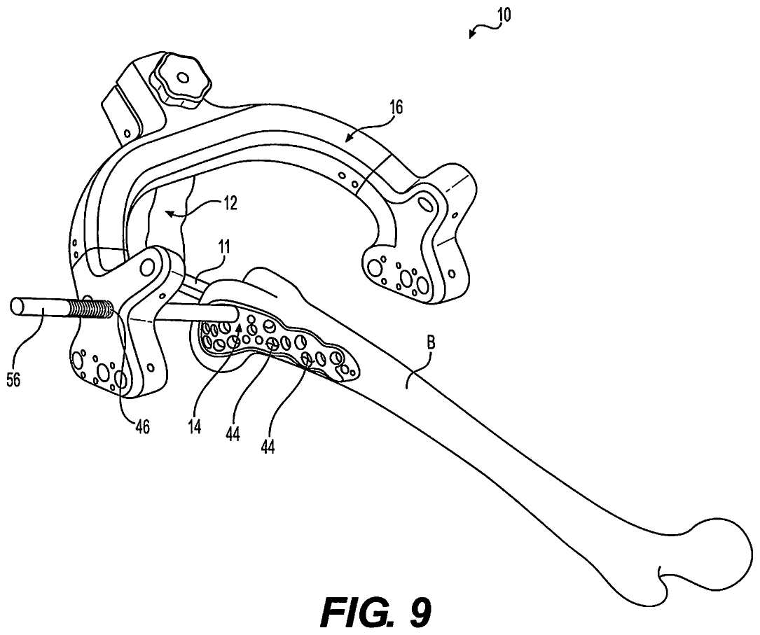

As shown in FIGS. 7 and 8, the bone plate 14 may include or define one or more plate openings 44 extending therethrough that are sized, shaped, and/or configured to receive one or more stabilizing fasteners therethrough. The plate openings 44 may be arranged or spaced about the bone plate 14 to form an opening pattern that enables the stabilizing fasteners to be arranged or positioned in one or more desired stabilizing fastener patterns. The stabilizing fasteners may be extended through the plate openings 44, for example, to be in a stabilizing fastener pattern that immobilizes one or more fracture fragments, stabilizes the bone B, and/or otherwise treats a bone fracture. In some examples, as shown in FIG. 9, the bone plate 14 may be positioned and/or oriented such that the trajectory of one or more plate openings 44 substantially matches or are coaxial with one or more nail openings 18 of the intramedullary nail 11 so that the bone plate 14 and the intramedullary nail 11 may be interlocked together. The bone plate 14 may be attached with locking or non-locking screws using either freehand technique or using an aiming guide (e.g., aiming guide 16).

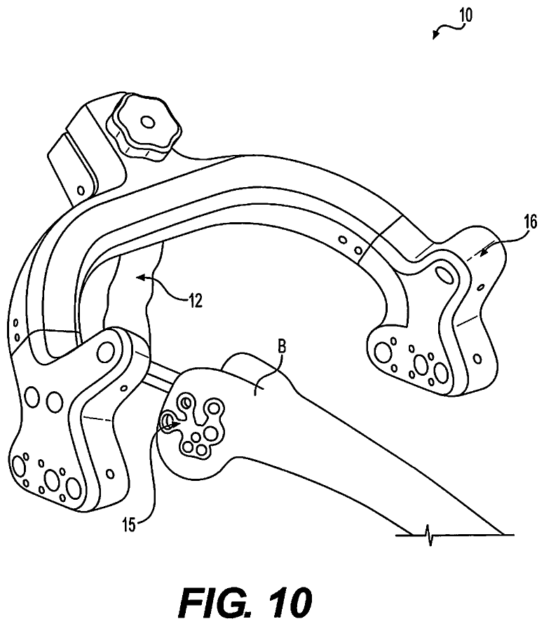

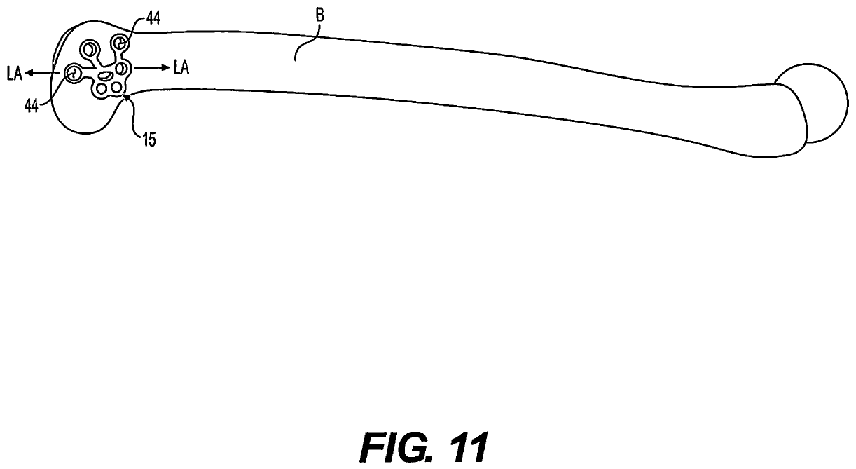

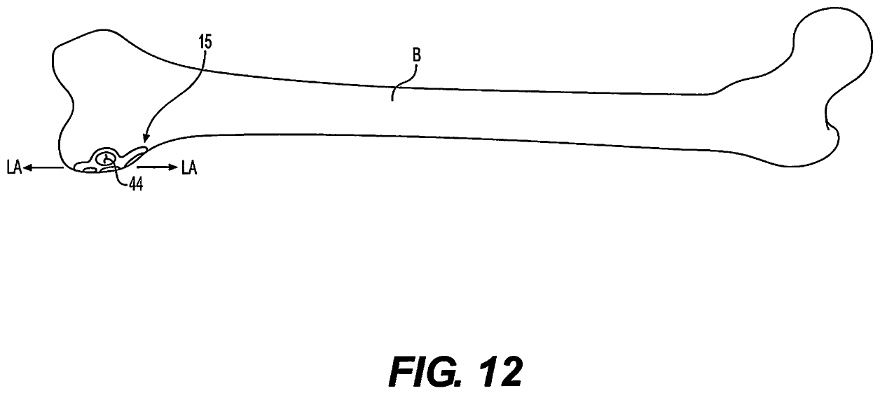

FIGS. 10-12 show another example bone plate 15 that may be used in the system 10 for treating a fracture in the bone B. Like bone plate 14, shown in FIGS. 7-9, the bone plate 15 may be oriented to extend generally along or parallel to the longitudinal axis LA of the bone B and be positioned on a medial and/or lateral side of the bone B to engage at least a portion of a condyle and/or shaft of the bone B. While bone plate 15 has a different shape and/or configuration than that of bone plate 14, bone plate 15 may be used with one or more stabilizing fasteners, intramedullary nail 11, insertion tool 12, and aiming guide 16 in the same or a substantially similar manner.

Referring to FIGS. 1, 9, and 13-14, the aiming guide 16 may include or define one or more targeting openings 46 extending therethrough that are sized, shaped, and/or configured to receive one or more stabilizing fasteners therethrough. The aiming guide 16 is configured to enable a user (e.g., a surgeon) to target or locate one or more nail openings 18 of the intramedullary nail 11 for inserting stabilizing fasteners without fluoroscopy. For example, the targeting openings 46 may be arranged or spaced about the aiming guide 16 to form an opening pattern that enables the stabilizing fasteners to be arranged or positioned in one or more desired stabilizing fastener patterns. The stabilizing fasteners may be extended through the targeting openings 46, for example, to be in a stabilizing fastener pattern that immobilizes one or more fracture fragments, stabilizes the bone B, and/or otherwise treats a bone fracture. In some examples, one or more targeting openings 46 may be used for plate-nail targeting, washer-nail targeting, and/or blocking screw insertion. For example, blocking screws may be extended through one or more targeting openings 46 to guide the trajectory of stabilizing fasteners to gain an anatomic reduction prior to final fixation.

In some examples, the aiming guide 16 may include a body and a pair of legs extending from the body. As shown in FIGS. 1, 9, 13, and 15, the aiming guide 16 may have or include a wishbone-shaped configuration. Each leg may include one or more targeting openings 46 such that targeting openings 46 may be positioned on opposite sides of the bone B, such as medial and lateral sides. In some examples, the aiming guide 16 may be fabricated from one or more composite, radiolucent material. In this manner, the aiming guide 16 will not obstruct the view of the intramedullary nail 11 and/or bone B when using fluoroscopy due to its wishbone-shaped configuration and/or radiolucent material.

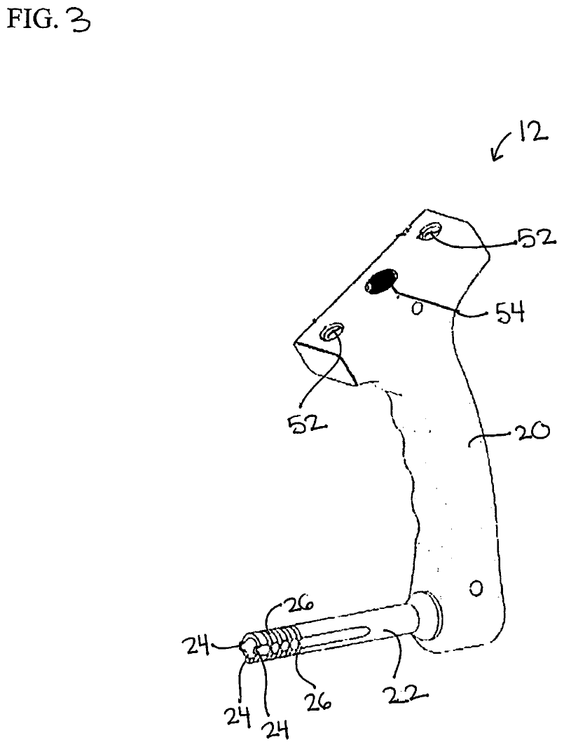

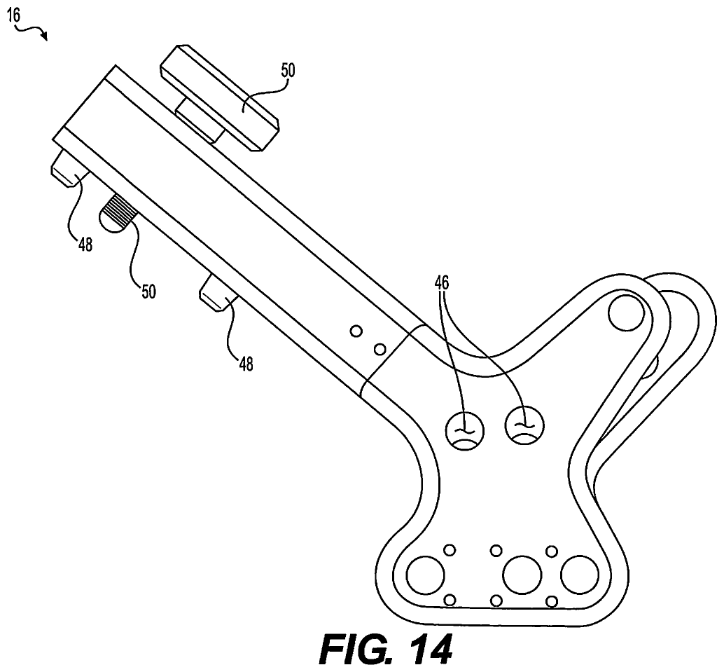

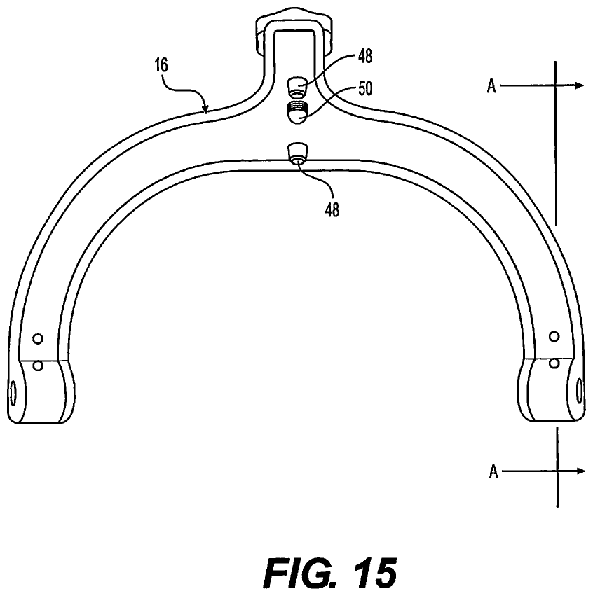

As shown in FIGS. 14 and 15, the body of the aiming guide 16 may include one or more locating pins 48 and/or a threaded knob 50 that enables the aiming guide 16 to be desirably positioned and/or oriented relative to insertion tool 12. In some examples, the locating pins 48 are sized, shaped, and/or configured to be received in one or more locating pin openings 52 (shown in FIGS. 3 and 4) defined in an upper portion of the handle 20, and/or the threaded knob 50 is sized, shaped, and/or configured to be received in a threaded insert 54 (shown in FIGS. 3 and 4) defined in the upper portion of the handle 20. The threaded knob 50 may be used to securely couple the aiming guide 16 to the insertion tool 12. Additionally, or alternatively, the insertion tool 12 may include one or more locating pins 48 and/or the threaded knob 50, and/or the aiming guide 16 may include the one or more locating pin openings 52 and/or threaded insert 54 defined therein.

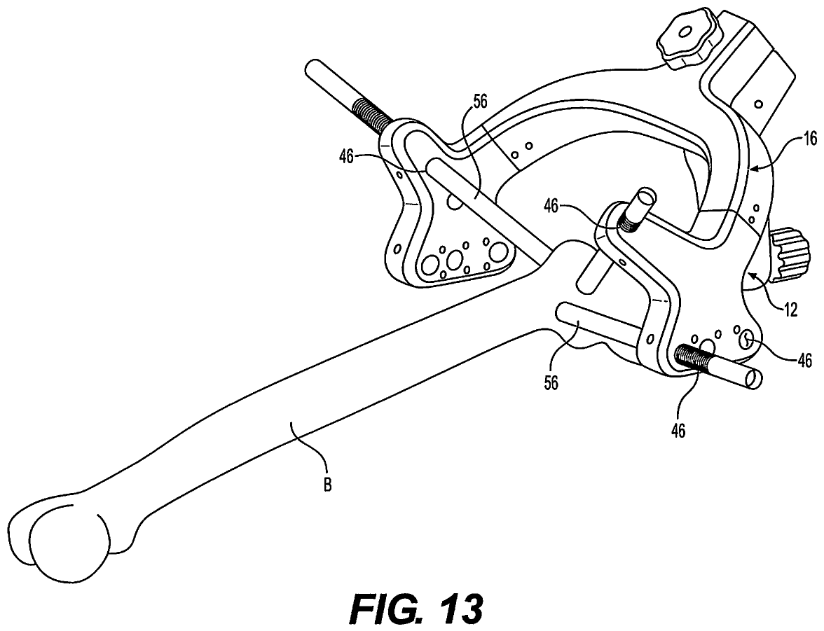

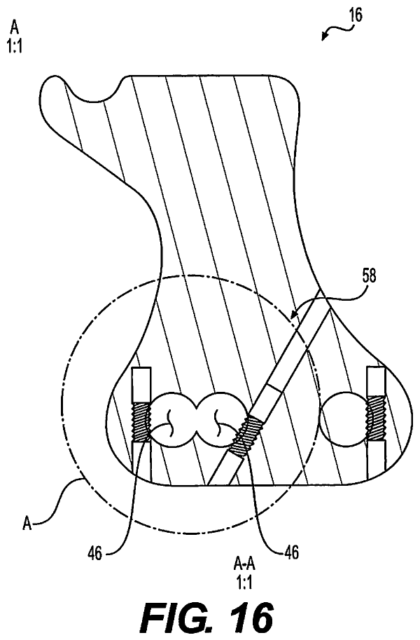

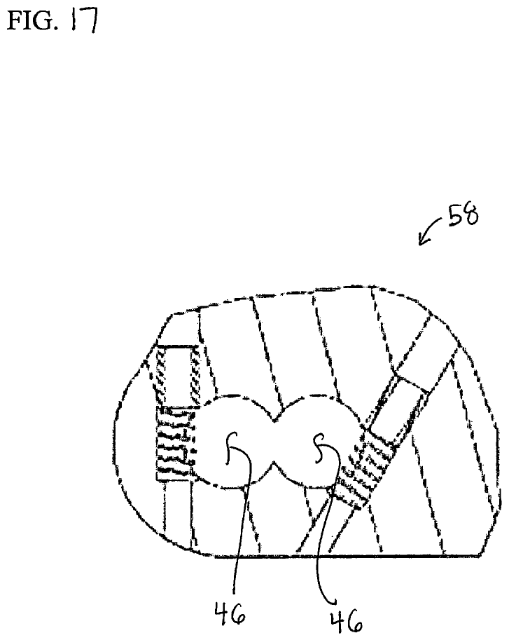

In some examples, as shown in FIGS. 9 and 13, one or more driver sleeves 56 may be extended through the targeting openings 46 for aligning and/or orienting the stabilizing fasteners. As shown in FIGS. 16 and 17, the aiming guide 16 may include or define one or more retention mechanisms 58 configured to retain one or more driver sleeves 56 in the targeting openings 46. The retention mechanism 58 may include, for example, a peak rod configured to control a movement of the driver sleeves 56. The peak rod may extend at least partially across the targeting opening 46 of the aiming guide 16 and may use deflection, due to material properties of the peak rod, to control movement of the one or more stabilizing fasteners. In some examples, the retention mechanism 58 of the aiming guide 16 includes a spring retention system that prevents a driver sleeve 56 from moving or falling out of the targeting openings 46. As shown in FIGS. 16 and 17, the spring retention system may include at least one spring configured to retain the driver sleeves 56. The springs may be retained in a pocket that is off center relative to the targeting opening 46 and may be configured to deflect when a driver sleeve 56 is inserted into the targeting openings 46 and exert a retaining force on an outer diameter of the driver sleeve 56 to prevent the driver sleeve 56 from falling out or slipping out of position. When the driver sleeve 56 is inserted into the targeting opening 46, the spring deflects and applies a retaining force to the outside of the driver sleeve 56 to prevent movement.

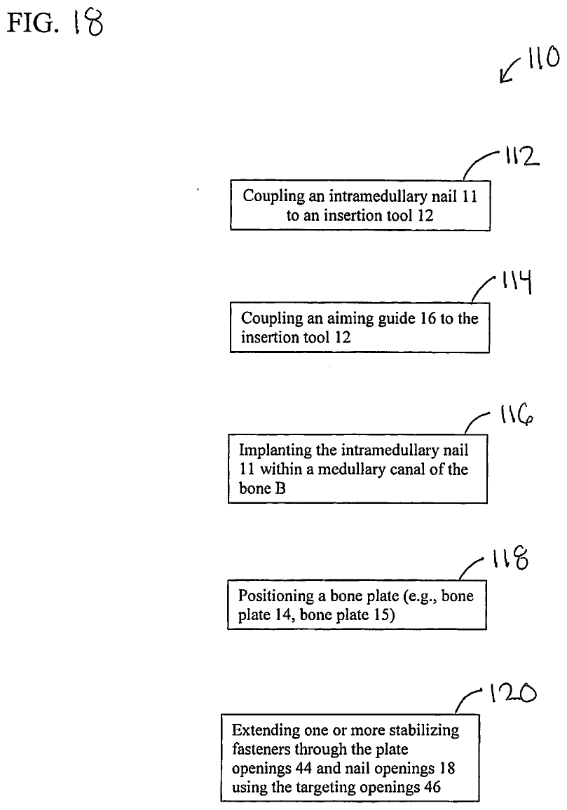

Referring to FIG. 18, a method 110 for treating a fracture in a bone B includes coupling an intramedullary nail 11 to an insertion tool 12 at operation 112. The intramedullary nail 11 includes one or more nail openings 18 extending therethrough for receiving one or more stabilizing fasteners.

An aiming guide 16 is coupled to the insertion tool at operation 114. The aiming guide 16 includes one or more targeting openings 46 for receiving the stabilizing fasteners.

The intramedullary nail 11 is implanted within a medullary canal of the bone B at operation 116. With the aiming guide 16 coupled to the insertion tool 12, the aiming guide 16 is positioned and/or oriented such that the trajectory of one or more targeting openings 46 substantially matches or are coaxial with one or more nail openings 18 of the intramedullary nail 11.

A bone plate (e.g., bone plate 14, bone plate 15) is positioned at operation 118. The bone plate includes one or more plate openings 44 extending therethrough. The bone plate may be positioned such that the plate openings 44 are axially aligned with the targeting openings 46. By positioning the bone plate in this manner, the trajectory of the plate openings 44 substantially match or are coaxial with one or more nail openings 18.

One or more stabilizing fasteners are extended through the plate openings 44 and nail openings 18 using the targeting openings 46 at operation 120. The insertion tool 12 facilitates positioning the intramedullary nail 11 to aid in securing the bone B while the stabilizing fasteners are being guided into the plate openings 44 and/or nail openings 18. The stabilizing fasteners may be used to interlock the intramedullary nail 11 and bone plate to compress the condyle of the bone B. In this manner, at least some of the load may be distributed along the bone plate instead of being concentrated at the bone B and/or intramedullary nail 11, mitigating trauma to the bone and/or decreasing recovery time.

Examples described herein may be used to treat one or more bone fractures. When an insertion tool is coupled to an aiming guide, a user (e.g., a surgeon) may extend one or more stabilizing fasteners at least partially through an intramedullary nail implanted within a medullary canal of a bone with the naked eye (e.g., without fluoroscopy) by extending the stabilizing fasteners through the targeting openings of the aiming guide. In some examples, a bone plate may be positioned by aligning the plate openings of the bone plate with the targeting openings of the aiming guide. In this manner, the user may extend one or more stabilizing fasteners through the targeting openings of the aiming guide to extend the stabilizing fasteners through the plate openings of the bone plate and the nail openings of the intramedullary nail with the naked eye (e.g., without fluoroscopy).

When introducing elements of the present disclosure or the one or more embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above apparatuses, systems, and methods without departing from the scope of the invention, it is intended that all matter contained in the above description and shown in the accompanying drawings shall be interpreted as illustrative and not in a limiting sense.

What is claimed:

1. An aiming guide for treating a fracture in a bone, the aiming guide comprising:
a body; and
a pair of legs extending from the body, each leg of the pair of legs including one or more targeting openings for receiving one or more stabilizing fasteners therethrough, the one or more targeting openings spaced about the pair of legs such that the aiming guide is positionable to axially align the one or more targeting openings with one or more openings of an intramedullary nail implanted within a medullary canal of the bone.

2. The aiming guide of claim 1, wherein the body includes one or more locating pins for use in coupling the aiming guide to an insertion tool.

3. The aiming guide of claim 1, wherein the body includes a threaded knob for use in coupling the aiming guide to an insertion tool.

4. The aiming guide of claim 1, further comprising one or more retention mechanisms configured to control a movement of one or more driver sleeves extended through the one or more targeting openings.

5. An aiming guide for treating a fracture in a bone, the aiming guide comprising:
one or more targeting openings for receiving one or more stabilizing fasteners;
one or more targeting openings spaced about the aiming guide such that the aiming guide is positionable to axially align the one or more targeting openings with one or more plate openings and the one or more nail openings for extending the one or more stabilizing fasteners therethrough when an intramedullary nail is implanted within the medullary canal of the bone.

6. The aiming guide of claim 5, further comprising an insertion tool coupleable to the intramedullary nail and the aiming guide, wherein the aiming guide is positioned such that the one or more targeting openings are axially aligned with the one or more nail openings when the insertion tool is coupled to the intramedullary nail and the aiming guide.

7. The aiming guide of claim 6, wherein the aiming guide includes one or more locating pins and the insertion tool includes one or more locating pin openings defined therein for receiving the one or more locating pins.

8. The aiming guide of claim 6, wherein the aiming guide includes a threaded knob and the insertion tool includes a threaded insert defined therein for receiving the threaded knob.

9. The aiming guide of claim 5, wherein the aiming guide has a wishbone-shaped configuration including a first leg and a second leg, a first targeting opening of the one or more targeting openings extending through the first leg, a second targeting opening of the one or more targeting openings extending through the second leg.

10. The aiming guide of claim 5, wherein the aiming guide includes one or more retention mechanisms configured to control a movement of one or more driver sleeves extended through the one or more targeting openings.
